(19) United States
(12) Patent Application Publication
(10) Pub. No.: US 2020/0338141 A1
(43) Pub. Date: Oct. 29, 2020

(54) Composition for Reducing Blood Uric Acid Level and Composition for Preventing or Improving Hyperuricemia, Pharmaceutical Composition, Food/Beverage Composition, Method for Reducing Blood Uric Acid Level, and Method of Preventing or Improving Hyperuricemia

(71) Applicant: MORINAGA MILK INDUSTRY CO., LTD., Tokyo (JP)

(72) Inventors: Mai Murata, Kanagawa (JP); Junichi Minami, Kanagawa (JP)

(73) Assignee: MORINAGA MILK INDUSTRY CO., LTD., Tokyo (JP)

(21) Appl. No.: 16/394,378

(22) Filed: Apr. 25, 2019

(51) Int. Cl. A61K 35/745; A61K 9/00; A61P 7/00

(52) U.S. Cl. CPC A61K 35/745; A61P 7/00; A61K 9/0095; A61K 9/0053

(57) ABSTRACT

It is an aspect to provide a novel composition for lowering blood uric acid levels and a novel composition for preventing or ameliorating hyperuricemia. A composition for lowering blood uric acid levels comprising Bifidobacterium breve as an active ingredient is provided. A composition for preventing or ameliorating hyperuricemia comprising Bifidobacterium breve as an active ingredient is provided. A composition for lowering blood uric acid levels and a composition for preventing or ameliorating hyperuricemia can be used in a pharmaceutical composition or a food or beverage composition.

### TECHNICAL FIELD

The present technology relates to a composition for lowering blood uric acid level, a composition for preventing or ameliorating hyperuricemia, a pharmaceutical composition or food or beverage composition including these compositions, a method for lowering blood uric acid level, and a method for preventing or ameliorating hyperuricemia.

### BACKGROUND ART

In humans, uric acid is an end product of the metabolism of ATP, which is an in vivo energy source, nucleic acids, and purines derived from food and drink. It is excreted primarily from the kidneys. When various factors acting on the pathway from uric acid production to excretion cause production of uric acid to become excessive or cause the amount excreted to decrease, the level of uric acid in the blood increases and hyperuricemia occurs. Known factors include obesity, overeating, alcohol consumption, excessive exercise (anaerobic exercise), and stress. Gout develops when hyperuricemia persists and crystallization and precipitation of uric acid occurs in joints and the kidneys. In recent years, the number of gout patients has increased in Japan due to lifestyle changes such as in eating habits, and the number of hyperuricemia patients, which are candidates for gout, has also increased.

Under these circumstances, the development of a composition or drug that reduces blood uric acid levels and prevents or ameliorates hyperuricemia is desired. There are two known types of therapeutic agents for hyperuricemia, namely, uric acid production inhibitors that prevent uric acid from being produced and uric acid excretion promoters that cause uric acid to be excreted. The former suppress uric acid formation by inhibiting xanthine oxidase (XO), which is an enzyme required in the decomposition of purine into uric acid. Meanwhile, the latter promote uric acid excretion by inhibiting a uric acid transporter molecule present in the proximal tubules of the kidneys known as URAT1 which performs uric acid reabsorption.

Food and beverage products or supplements that can be taken on a daily basis are desired, and lactic acid bacteria and bifidobacteria have garnered attention as active ingredients. Patent Document 1 discloses an agent for preventing or ameliorating hyperuricemia which includes Bifidobacterium longum BB536 (ATCC BAA-999) as an active ingredient. Patent Document 2 discloses a blood uric acid value lowering agent which includes a specific type of lactic acid bacteria or bifidobacteria as an active ingredient.

### CITATION LIST

### Patent Literature

Patent Document 1: JP 2009-143820 A

Patent Document 2: JP 2017-031102 A

### SUMMARY

While various methods for preventing and treating hyperuricemia have been proposed using synthetic compounds, there are concerns about side effects. Several methods for preventing and treating hyperuricemia have also been reported that entail ingestion of probiotic bacteria and fermentation products, such as those in Patent Documents 1 and 2. However, different strains of probiotics have been known to exhibit different physiological effects, and consistent results have not been obtained.

It is the primary aspect of the present technology to provide a novel composition for lowering blood uric acid levels and a novel composition for preventing or ameliorating hyperuricemia.

The present technology provides a composition for lowering blood uric acid levels comprising Bifidobacterium breve as an active ingredient. The present technology also provides a composition for preventing or ameliorating hyperuricemia comprising Bifidobacterium breve as an active ingredient. Here, the Bifidobacterium breve can be Bifidobacterium breve MCC1274 (FERM BP-11175). These compositions can be used as a pharmaceutical composition or as a food or beverage composition.

The present technology also provides use of Bifidobacterium breve in a hyperuricemia preventing or ameliorating agent, hyperuricemia preventing or ameliorating medicine, or hyperuricemia preventing or ameliorating food or beverage product. Here, the Bifidobacterium breve can be Bifidobacterium breve MCC1274 (FERM BP-11175). The present technology also provides blood uric acid level lowering method comprising administering Bifidobacterium breve to a subject, and a hyperuricemia preventing or ameliorating method comprising administering Bifidobacterium breve to a subject. Here, the Bifidobacterium breve can be Bifidobacterium breve MCC1274 (FERM BP-11175).

It is an aspect of the present invention to provide a composition for lowering blood uric acid levels comprising Bifidobacterium breve as an active ingredient.

It is a further aspect of the present invention to provide a composition for preventing or ameliorating hyperuricemia comprising Bifidobacterium breve as an active ingredient.

It is a further aspect of the present invention to provide a composition as described above, wherein the Bifidobacterium breve is Bifidobacterium breve MCC1274 (FERM BP-11175).

It is a further aspect of the present invention to provide a composition as described above, wherein the composition is a pharmaceutical composition.

It is a further aspect of the present invention to provide a composition as described above, wherein the composition is a food or beverage composition.

It is a further aspect of the present invention to provide a composition as described above, wherein the composition comprises from 10⁶ to 10¹² cfu of Bifidobacterium breve MCC1274 (FERM BP-11175) per packaging unit.

It is a further aspect of the present invention to provide a composition as described above, wherein the composition comprises from 10⁶ to 10¹² cfu of Bifidobacterium breve MCC1274 (FERM BP-11175) per serving.

It is a further aspect of the present invention to provide a composition as described above, wherein the composition is fermented milk.

It is a further aspect of the present invention to provide use of Bifidobacterium breve in a blood uric acid level lowering agent, blood uric acid level lowering medicine, or blood uric acid level food or beverage product.

It is a further aspect of the present invention to provide use of Bifidobacterium breve in a hyperuricemia preventing or ameliorating agent, hyperuricemia preventing or ameliorating medicine, or hyperuricemia preventing or ameliorating food or beverage product.

It is a further aspect of the present invention to provide a use as described above, wherein the Bifidobacterium breve is Bifidobacterium breve MCC1274 (FERM BP-11175).

It is a further aspect of the present invention to provide a method for lowering blood uric acid level comprising administering Bifidobacterium breve to a subject.

It is a further aspect of the present invention to provide a method for preventing or ameliorating hyperuricemia comprising administering Bifidobacterium breve to a subject.

It is a further aspect of the present invention to provide a method as described above, wherein the Bifidobacterium breve is Bifidobacterium breve MCC1274 (FERM BP-11175). The present technology is able to provide a novel blood uric acid level lowering composition and hyperuricemia preventing or ameliorating composition. The present technology is not limited to the effect described here but also includes any other effect mentioned herein.

### DETAILED DESCRIPTION

The following is a description of particular embodiments of the present technology. The embodiments described below are typical examples of embodiments of the present technology and should not be interpreted as narrowing the scope of the present technology in any way.

1. Blood Uric Acid Level Lowering Composition and Hyperuricemia Preventing or Ameliorating Composition

A blood uric acid level lowering composition and hyperuricemia preventing or ameliorating composition of the present technology (“compositions of the present technology” below) are characterized by the use of Bifidobacterium breve.

Bifidobacterium breve (Bifidobacterium breve) is only one of the bacterial species belonging to the genus Bifidobacterium. Bifidobacterium breve lives primarily in the large intestine of infants, and is known among the bacteria belonging to the genus Bifidobacterium as an infant-type Bifidobacterium along with others such as Bifidobacterium longum subsp. infantis.

Because the active ingredient in a composition of the present technology is Bifidobacterium breve, which primarily resides in the large intestine of infants and children, it is safe and effective and side effects are not a concern even during long-term, continuous administration. It can also be safely combined with other drugs.

When the blood uric acid level decreases suddenly, uric acid crystals that have accumulated in the joints may dissolve at once, causing an attack of gout. Therefore, when treating hyperuricemia, blood uric acid levels should not be lowered over a short period of time but rather gradually over a longer period of time, followed by maintenance of a target value over a long period of time. Because a composition of the present technology can be taken safely over a long period of time, it can be used for such a purpose.

Bacteria belonging to Bifidobacterium breve include Bifidobacterium breve MCC1274 (FERM BP-11175), M-16V (NITE BP-02622), UCC2003, YIT4010, YIT4064, BBG-001, BR-03, B632 (DSMZ24706), C50, Bb99 (DSM13692), R0070, ATCC15700, ATCC15698, and DSM24732. Among these, use of Bifidobacterium breve MCC1274 (FERM BP-11175) is a particular example in the present technology.

Bifidobacterium breve MCC1274 was deposited on Aug. 25, 2009 under Accession Number IPOD FERM BP-11175 at the National Institute of Technology and Evaluation-International Patent Organism Depositary (NITE-IPOD), Central 6, 1-1-1, Higashi, Tsukuba, Ibaraki, Japan 305-8566 (Current location: Room 120, 2-5-8, Kazusakamatari, Kisarazu, Chiba 292-0818, Japan), and can be obtained from this organization.

Note that the aforementioned bacteria name is not limited to the strain that was deposited and registered at the aforementioned organization under this name, but also includes substantially equivalent strains (referred to as “derived strains” or “induced strains”). Specifically, “MCC1274 (FERM BP-11175)” is not limited to the strain deposited at the depositary under the accession number for MCC1274 (FERM BP-11175), but includes all substantially equivalent strains. Here, “a strain substantially equivalent to the deposited strain” means a strain which belongs to the same species as the deposited strain and from which the sleep-promoting effect that is an effect of the present technology can be obtained. A strain substantially equivalent to the deposited strain can be, for example, a derivative strain having the deposited strain as the parent strain. Derivative strains include strains bred from deposited strains and strains produced naturally from deposited strains.

The substantially equivalent strains and derivative strains include the following:

(1) Strains deemed as identical to the deposited strain using the randomly amplified polymorphic DNA (RAPD) method and the pulsed-field gel electrophoresis (PFGE) method (see Probiotics in food/health and nutritional properties and guidelines for evaluation 85, Page 43).

(2) Strains having only genes derived from the deposited strain, no foreign genes, and a DNA identity of 95% or more with the deposited strain.

(3) Strains bred from the deposited strain (including strains bred by genetic engineering modification, mutation, and spontaneous mutation) and having the traits of the deposited strain.

The term “alleviation” means reversal of a symptom or disease, prevention or delay in deterioration of a symptom or disease, reversal, prevention or delay in the progression of a symptom or disease, or treatment of a symptoms or disease. Here, “prevention” means preventing or delaying the onset of a symptom or disease in a subject, or reducing the risk of developing a symptom or disease in a subject.

A composition of the present technology has an effect of lowering blood uric acid levels as demonstrated in the examples described below. In the present technology, “hyperuricemia” includes symptoms of gout and urolithiasis caused by hyperuricemia.

There are no particular restrictions on the subjects receiving a composition of the present technology. They can be any animal including humans. There are no particular sex or age restrictions. Examples include those who consume too much alcohol or purine from their diet, men who do not eat enough vegetables, and physically inactive adults. Because a composition of the present technology is safe, it can be taken by infants and children, pregnant, perinatal and lactating women, and elderly persons and patients at high risk of hyperuricemia.

The Bifidobacterium breve MCC1274 (FERM BP-11175) serving as the active ingredient in a composition of the present technology may be a culture including Bifidobacterium breve MCC1274 (FERM BP-11175).

There are no particular restrictions on the medium for culturing the Bifidobacterium breve used in the present technology. It can be any medium commonly used for culturing bacteria belonging to the genus Bifidobacterium.

The carbon source can be saccharides such as glucose, galactose, lactose, arabinose, mannose and sucrose, starches, starch hydrolysates, and waste molasses depending on assimilability. Ammonia, ammonium salts such as ammonium sulfate, ammonium chloride and ammonium nitrate, and nitrates can also be used as the carbon source. Inorganic salts that can be used include sodium chloride, potassium chloride, potassium phosphate, magnesium sulfate, calcium chloride, calcium nitrate, manganese chloride, and ferrous sulfate. In addition, organic components such as peptones, soybean powders, defatted soybean meal, meat extracts, and yeast extracts can be used.

There are no particular restrictions on the culturing conditions as long as the effect of the present technology is not impaired. The culture temperature is usually from 25 to 50° C., and or from 35 to 42° C. The culture can be conducted under anaerobic conditions. For example, it can be cultivated while supplying an anaerobic gas such as a carbon dioxide gas. However, the culture may also be cultivated under microaerophilic conditions, such as in a stationary liquid culture.

The Bifidobacterium breve used in the present technology may be used in the form of the resulting culture with or without dilution or concentration, or the bacteria may be collected from the resulting culture and used. The term “culture” includes the culture supernatant.

The bacteria may be used in the resulting culture with or without dilution or concentration, or the bacteria may be collected from the resulting culture and used. Other operations such as heating and freeze-drying can be performed after culturing as long as the effects of the present technology are not impaired. The bacteria may be alive or dead. Live bacteria can be subjected to the bacterial solution freezing method, the spray drying method, the freeze-drying method, or the oil drop method. Dead bacteria may be sterilized by heat or freeze-drying. Other methods that can be used to prepare dead bacteria include the spray drying method (spray dry method), retort sterilization method, freeze-drying method, UHT sterilization method, pressure sterilization method, high pressure steam sterilization method, dry heat sterilization method, distributed steam disinfection method, electromagnetic wave sterilization method, electron beam sterilization method, high frequency sterilization method, radiation sterilization method, UV sterilization method, ethylene oxide gas sterilization method, hydrogen peroxide gas plasma sterilization method, and chemical sterilization method (alcohol sterilization method, formalin fixation method, electrolytic water treatment method). The bacteria may also be disrupted. The bacteria can be disrupted while live or dead, and heated or freeze-dried after disruption. The disruption can be performed using physical crushing, enzyme dissolution, chemical processing, or autolysis using methods and equipment common in the art.

Physical disruption may be performed in the form of a suspension or powder. The physical disruption can be performed by agitation using an ultrasonic homogenizer, homogenizer, ball mill, bead mill, dyno mill or planetary mill, under pressure using a jet mill, French press or cell disruptor, or by cellular damage using filtration. In an enzyme dissolution treatment, an enzyme such as lysozyme can be used to disrupt the cell structure of the lactic acid bacterial cells. In chemical treatment, a surfactant such as a soybean phospholipid or glycerin fatty acid ester can be used to disrupt the cell structure of lactic acid bacterial cells. In autolysis, lactic acid bacterial cells can be dissolved using some of the enzymes of the lactic acid bacteria themselves. In the present technology, physical disruption is preferred because other chemicals and compounds do not have to be added.

A composition of the present technology can be the active ingredient alone or a composition containing any ingredient along with the active ingredient. There are no particular restrictions on the other ingredients and can be any additive commonly used in pharmaceutical products (such as the pharmaceutical carriers described later).

2. Specific Forms of Compositions in the Present Technology

A composition of the present technology can assume the form of a food or beverage product, a pharmaceutical product, a quasi-drug, or a feed.

The purpose of the present embodiment can be therapeutic or non-therapeutic. A “non-therapeutic purpose” is a concept that does not include the practice of medicine, that is, the treatment of the human body with therapy. Examples include the promotion of health and enhancement of beauty.

Food and Beverage Products

A composition of the present technology can be added to an existing food or beverage product or mixed with the ingredients of a food or beverage product to prepare a novel food or beverage product.

Food or beverage products using a composition of the present technology can be in liquid, paste, solid, or powdered form. In addition to tablet cakes and liquid foods, examples include commercially available products such as flour products, instant foods, agricultural products, fish products, meat products, milk and dairy products, fats and oils, basic seasonings, mixed seasonings/foodstuffs, frozen foods, confectioneries, and beverages.

Examples of flour products include breads, macaroni, spaghetti, noodles, cake mixes, fried flour, and bread crumbs. Instant foods include instant noodles, cup noodles, retort packaged foods, canned foods, microwave foods, instant soups, instant miso soups, canned soup, and freeze-dried foods. Agricultural products such as canned agricultural products, canned fruits, jams and marmalades, pickles, boiled beans, dry agricultural products, and cereals (processed grain products). Fish products include canned fish, fish filets and sausages, fish pastes, seafood delicacies, and tsukudani. Meat products include canned meat and pastes, and meat filets and sausages. Milk and dairy products include fermented milk products such as yogurt, processed milk, milk beverages, lactic acid bacteria beverages, cheeses, ice creams, powdered milks, creams, powdered milk formulas for children, infant food supplements, and mother's milk for pregnant women and nursing women. Oils and fats include butters, margarines, and vegetable oils. Examples of basic seasonings include soy sauce, miso, sauces, tomato-based seasonings, mirin, and vinegar. Examples of mixed seasonings/foodstuffs include cooking mix, curry ingredients, sauces, dressings, noodles, and spices. Examples of freeze-dried foods include raw frozen foods, semi-cooked frozen foods, and cooked frozen foods. Confectioneries include caramels, candies, chewing gum, chocolates, cookies, biscuits, cakes, pies, snacks, crackers, Japanese sweets, rice cakes, bean candies, and dessert sweets. Examples of beverages include soft drinks, natural juices, juices, soft drinks containing juices, broths, fruit drinks with berries, vegetable drinks, soymilk, soy milk beverages, coffee drinks, tea drinks, powdered beverages, concentrated beverages, sports drinks, health drinks, and alcoholic beverages. Other commercially available food products include baby foods, sprinkled-on seasonings, and chazuke drinks.

A food or beverage composition of the present technology can be prepared by adding the bacteria to the raw materials of a food or beverage product, or prepared in the same manner as an ordinary food or beverage product except for the addition of the bacteria. The bacteria can be added at any stage of the food or beverage preparation process. A food or beverage product may also be prepared via a fermentation step using the bacteria. Examples of such food or beverage products include lactic acid bacteria drinks and fermented milk products.

The raw materials used in these food and beverage compositions can be any raw material commonly used in food and beverage products. The resulting food or beverage composition can be ingested orally.

Also, the bacteria can be added to pumped breast milk for oral ingestion by newborns and infants or for ingestion by nasogastric feeding tube.

A food or beverage composition of the present technology can be a component known or likely to be found to have a probiotic effect or a component that assists with a probiotic effect, as long as the effects of the present technology are not impaired.

Examples include proteins such as whey protein, casein protein, soy protein and pea protein (pea protein), as well as mixtures and degradation products thereof; amino acids such as leucine, valine, isoleucine or glutamine; vitamins such as vitamin B6 or vitamin C; creatine; citric acid; fish oils; and oligosaccharides such as isomaltooligosaccharides, galactooligosaccharides, xylooligosaccharides, soybean oligosaccharides, fructooligosaccharides, lactulose, and human milk oligosaccharides (HMO) combined with the bacteria.

Human milk oligosaccharides that can be used include neutral human milk oligosaccharides such as 2′-fucosyl lactose, 3-fucosyl lactose, 2′,3-difucosyllactose, lacto-N-triose II, lacto-N-tetraose, lacto-N-neotetraose, lacto-N-fucopentaose I, lacto-N-neofcopentaose, lacto-N-fucopentaose II, lacto-N-fucopentaose III, lacto-N-fucopentaose V, lacto-N-neofcopentaose V, lacto-N-difucohexaose I, lacto-N-difucohexaose II, 6′-galactosyl lactose, 3′-galactosyl lactose, lacto-N-hexaose, and lacto-N-neohexaose, as well as acidic human milk oligosaccharides such as 3′-sialyllactose, 6′-sialyl lactose, 3-fucosyl-3′-sialyl lactose, and disialyl-lacto-N-tetraose.

The amount of Bifidobacterium breve MCC1274 (FERM BP-11175) in a food or beverage product according to the present technology can be freely selected as long as the effects of the present technology are not impaired. In the present technology, the amount of Bifidobacterium breve MCC1274 (FERM BP-11175) in a food or beverage product is preferably from 1×10³ to 1×10¹² cfu/g of the final composition of the food or beverage product. In terms of the administered dose, it is at least 1×10³ cfu per day, preferably at least 1×10⁶ cfu per day, more preferably at least 1×10⁸ cfu per day, and even more preferably at least 2×10¹⁰ cfu per day. In the present technology, a food or beverage product preferably comprises from 10⁶ to 10¹² cfu of Bifidobacterium breve MCC1274 (FERM BP-11175) per serving. Here, cfu means colony forming unit. In the case of dead bacteria, cfu/g or cfu/ml can be converted to cells/g or cells/ml. In the case of disrupted bacteria, the number of cells (cells/g) prior to disruption can be indicated in terms of weight.

Functional Health Foods and Patient Foods

A composition according to the present technology can also be applied to functional health foods and patient foods. Because functional health foods are administered in the form of a tablet, capsule, or ordinary food product depending on market trends at home and abroad and in conformity with existing health food regulations, they are generally classified in one of three categories as specialized health foods, functionally labeled foods, or functional nutritional foods.

Specialized health foods are foods intended for people who cannot eat regular meals, such as sick people, infants, and elderly people. These include patient foods (subject to standard criteria and requiring individual approval), powdered milk formula for pregnant women and lactating women, infant formula, and food for people who have difficulty swallowing.

While not restricted to the following description, a composition of the present technology may be applied to low purine food products for those with elevated uric acid, low protein food products for those with impaired kidney function, personalized food products for patients, and functionally labeled food products for healthy persons and those with a normal to somewhat low uric acid index.

A food or beverage product according to the present technology can also be infant formula. Different types of infant formula include infant formula for infants from 0 to 12 months, follow-up milk for infants from 6 to 9 months and small children (up to 3 years old), low birth weight infant formula for newborns weighing less than 2,500 g at birth (low birth weight infants), and therapeutic milks used to treat children with conditions such as milk allergies and lactose intolerance.

Examples of food and beverage products according to the present technology include mother's milk (powdered milk formula including the right balance of nutrients for pregnant women and lactating women), nutritionally adjusted foods such as powdered milk formula for adults, dietary supplements, nutritive function foods such as liquid foods, and patient foods such as powdered milk with reduced phosphorus (special use food).

A powdered milk formula of the present technology can be prepared using the following method.

Specifically, the present technology provides a method for producing a powdered milk formula or mother's milk in which a powdered bacterium related to the genus Bifidobacterium is mixed with a prebiotic and/or milk powder to obtain a powdered milk for reducing blood uric acid levels or preventing or ameliorating hyperuricemia.

For example, the present technology is a method for manufacturing a powdered milk for fortifying mother's milk comprising steps (A) to (C) below:

(A) culturing the Bifidobacterium in a medium containing milk components to obtain a culture;

(B) spray drying and/or freeze-drying the culture to obtain a cell powder; and

(C) mixing the powdered bacteria with a prebiotic and/or powdered milk to obtain a powdered milk for reducing blood uric acid levels or preventing or ameliorating hyperuricemia.

Supplements

A food composition of the present technology can be a simple supplement for reducing blood uric acid levels or preventing or ameliorating hyperuricemia.

A supplement for reducing blood uric acid levels or preventing or ameliorating hyperuricemia can be produced using the following method.

Specifically, the present technology provides a method for producing a supplement for reducing blood uric acid levels or preventing or ameliorating hyperuricemia comprising steps (A) and (B) below:

(A) mixing a prebiotic, Bifidobacterium bacterium, and excipient together to obtain a mixture; and

(B) tableting the mixture.

Functionally Labeled Food and Beverage Products

A food or beverage product defined in the present technology can be provided and sold as a food or beverage product labeled by intended use (especially, health use) or function.

“Labeled” includes all acts performed in order to inform consumers of the intended use. Any expression used to evoke or infer the intended use, regardless of the purpose of the label, the content of the label, the object to be labeled, or the medium is considered a “labeling” act in the present technology.

A “label” uses expressions enabling the consumer to clearly identify the intended use. This includes descriptions of the intended use on the food or beverage product or packaging for the product which is displayed for shipment and delivery, descriptions of the intended use in price lists and business documents related to import and advertising, and descriptions of the intended use in information related to the food or beverage product provided using an electromagnetic method (via the internet, etc.).

The content of the label can be approved by the relevant government authorities (for example, a label approved by relevant governmental organizations and used in an approved manner). The label content can be included in packaging, containers, catalogs, brochures, and point-of-purchase (POP) advertising.

The labeling also indicates whether the product is a health food, functional food, patient food, enteral nutrition food, special purpose food, health-promoting food, specific health food, functional-labeled food, nutritional food, or quasi-drug. These include labels approved by consumer agencies in accordance with, for example, special health product procedures, function-claiming product procedures, and analogous procedures. Specific examples include labels indicating a specific health food, labels indicating a specific health food with conditions, labels indicating a function-claiming product, labels indicating effects on bodily structures and functions, and labels indicating reduced risk of disease. For typical examples see the labels for specific health foods (especially, the intended health effect) included in Ordinance for Enforcement of the Health Promotion Act (Ordinance No. 86 of the Japanese Ministry of Health, Labor and Welfare dated Apr. 30, 2003, and the labels for function-claiming products included in Food Labeling Act (Act No. 70 of 2013).

The wording used in these labels is not necessarily restricted to wording on the lowering of blood uric acid levels or on the prevention or amelioration of hyperuricemia. The scope of the present technology also includes wording regarding the prevention, treatment and/or amelioration of disorders related to the lowering of blood uric acid levels and the prevention or amelioration of hyperuricemia. For example, the wording may be based on intended uses which are recognized by consumers as having a preventative or ameliorative effect on blood uric acid levels or hyperuricemia, such as “for those who are concerned about blood uric acid levels,” “for those with high blood uric acid levels,” and “for those who are worried about hyperuricemia.” It can also be labeled as having a blood uric acid level lowering effect or preventing or ameliorating hyperuricemia based on results of an evaluation using a new blood uric acid level measuring method.

Pharmaceutical and Quasi-Drug Products

A composition for preventing or ameliorating a functional gastrointestinal disorder according to the present technology can be added to an existing pharmaceutical product or quasi-drug (“pharmaceutical product etc.” below) or mixed with the ingredients of a pharmaceutical product etc. to prepare a novel pharmaceutical product etc.

When a composition of the present technology is to be used in a pharmaceutical product etc., the pharmaceutical product etc. can be formulated in the desired dosage form depending on whether the method of administration is oral or parenteral. There are no particular restrictions on the dosage form. In the case of oral administration, the composition can be formulated in the form of a solid preparation such as a powder, granules, tablet, lozenge, or capsule; or a liquid preparation such as a solution, syrup, suspension, or emulsion. In the case of parenteral administration, the composition can be formulated as a suppository, spray, inhalant, ointment, patch, or injectable. In the present technology, formulation as an orally administered dosage form is preferred. It can be formulated in the desired dosage form using any method common in the art.

The composition may be compounded with a suitable carrier during formulation. In addition to a composition for preventing or ameliorating a functional gastrointestinal disorder according to the present technology, components ordinarily used in the formulation process include excipients, pH adjusters, coloring agents, and flavoring agents. Another component having a known or anticipated effect of preventing, ameliorating and/or treating a disease or symptom can be included if appropriate.

Depending on the dosage form, the carrier in the formulation can be an organic or inorganic carrier. Examples of carriers in solid formulations include excipients, binders, disintegrants, lubricants, stabilizers, and flavoring agents.

Examples of excipients include sugar derivatives such as lactose, sucrose, glucose, mannitol, and sorbite; starch derivatives such as corn starch, potato starch, α-starch, dextrin, and carboxymethyl starch; cellulose derivatives such as crystalline cellulose, hydroxypropyl cellulose, hydroxypropyl methylcellulose, carboxymethylcellulose, and carboxymethylcellulose calcium; gum arabic; dextran; pullulan; silicate derivatives such as light anhydrous silicic acid, synthetic aluminum silicate, and magnesium aluminometasilicate; phosphate derivatives such as calcium phosphate; carbonate derivatives such as calcium carbonate; and sulfate derivatives such as calcium sulfate.

In addition to the excipients mentioned above, examples of binders include gelatins, polyvinyl pyrrolidone, and macrogol.

In addition to the excipients mentioned above, examples of disintegrants include chemically modified starches such as croscarmellose sodium, carboxymethyl starch sodium, and crosslinked polyvinylpyrrolidone, as well as cellulose derivatives.

Examples of lubricants include talc; stearic acid; metallic stearates such as calcium stearate and magnesium stearate; colloidal silica; waxes such as Veegum and spermaceti; boric acid; glycol; carboxylic acids such as fumaric acid and adipic acid; carboxylic acid sodium salts such as sodium benzoate; sulfates such as sodium sulfate; leucine; lauryl sulfate such as sodium lauryl sulfate and magnesium lauryl sulfate; silicas such as anhydrous silicic acid and silicic acid hydrate; and starch derivatives.

Examples of stabilizers include para-hydroxybenzoic acid esters such as methylparaben and propylparaben; alcohols such as chlorobutanol, benzyl alcohol, and phenylethyl alcohol; benzalkonium chloride; acetic anhydride; and sorbic acid.

Examples of flavoring agents include sweetening agents, acidulants, and flavorings.

Solvents such as water and flavoring agents are examples of carriers used in solutions for oral administration.

The amount of Bifidobacterium breve MCC1274 (FERM BP-11175) in a pharmaceutical product etc. according to the present technology can be freely selected as long as the effects of the present technology are not impaired. In the present technology, the amount of Bifidobacterium breve MCC1274 (FERM BP-11175) in a pharmaceutical product etc. is preferably from 1×10³ to 1×10¹² cfu/g of the final composition of the pharmaceutical product etc. In terms of the administered dose, it is at least 1×10³ cfu per day, at least 1×10⁶ cfu per day, at least 1×10⁸ cfu per day, or at least 2×10¹⁰ cfu per day. In the present technology, a pharmaceutical product etc. can include from 10⁶ to 10¹² cfu of Bifidobacterium breve MCC1274 (FERM BP-11175) per packaging unit.

Feed

A composition of the present technology can be added to an existing feed or mixed with the ingredients of a feed to prepare a novel feed.

When a composition of the present technology is added to a feed, feed ingredients include cereals such as corn, wheat, barley, and rye; brans such as wheat bran, rice bran, and defatted bran; meals such as corn gluten meal and corn germ meal; animal products such as skimmed milk powder, whey, fish meal, and bone meal; yeasts such as beer yeast; minerals such as calcium phosphate and calcium carbonate; oils and fats; amino acids; and sugars. Examples of feeds include animal feed (pet food, etc.), livestock feed, and fish food.

The amount of Bifidobacterium breve MCC1274 (FERM BP-11175) in a feed according to the present technology can be freely selected as long as the effects of the present technology are not impaired. In the present technology, the amount of Bifidobacterium breve MCC1274 (FERM BP-11175) in a feed can be from 1×10³ to 1×10¹² cfu/g of the final composition of the feed. In terms of the administered dose, it is at least 1×10³ cfu per day, at least 1×10⁶ cfu per day, at least 1×10⁸ cfu per day, or at least 2×10¹⁰ cfu per day.

### EXAMPLES

The following is a more detailed description of the present technology with reference to examples. The examples described below are typical examples of the present technology and should not be interpreted as narrowing the scope of the present technology in any way.

### Example 1

Preparation of Test Sample

A fermented milk product containing Bifidobacterium breve MCC1274 (FERM BP-11175) was prepared using the following process. First, raw milk, water as needed, and other components were mixed together, and homogenization and heat sterilization were performed in the usual manner. Freeze-dried powder of Bifidobacterium breve MCC1274 (FERM BP-11175) and lactic acid bacterium starter were added (inoculated) in the heat-sterilized milk preparation, and fermentation was allowed to occur at a constant fermentation temperature. When the pH reached the target value, the curds were broken up by stirring and cooled to 10° C. or lower to obtain the fermented milk used as a test sample.

Test Subjects

Healthy subjects (with a BMI from 25 to 30) aged 20 to 65 years at the time of consent were registered in the clinical trial as subjects. In addition, 70 persons who did not violate exclusion criteria (1) to (6) below were selected as subjects based on body composition measurements, blood tests, and physician interviews. The average age of the subjects was 47.6±8.6 years.

(1) Persons with a medical history including serious disease

(2) Persons receiving medical treatment for lifestyle-related disease (diabetes, high blood pressure, dyslipidemia)

(3) Persons with drug allergies or serious food allergies

(4) Persons who are pregnant, intending to become pregnant during the trial period, or are breastfeeding

(5) Persons who are heavy smokers and/or heavy drinkers

(6) Persons deemed to be unsuitable as a test subject by the examining doctor or assisting doctor based on the subject's background, physical findings, or the interview.

Testing Methodology

After a two-week observational period, the subjects ingested the test sample once a day, morning, day or night, for 12 consecutive weeks. The number of viable Bifidobacterium breve MCC1274 (FERM BP-11175) bacterial in the test sample was at least 100 million per day (per unit). In other words, the daily intake of the test group was at least 100 million viable Bifidobacterium breve MCC1274 (FERM BP-11175) bacteria.

The subjects were given blood tests before taking the test sample (baseline: 0 weeks) and at 12 weeks to measure blood uric acid concentrations (mg/dL). After the start of testing, one person decided to discontinue participation. A review was conducted to exclude persons who did not meet analysis criteria established in advance (namely, those who had an ingestion rate of less than 80%, those who violated the medicine dose or ingested prohibited foods, and those who were found to have violated or deviated significantly from the test plan). However, none of them were excluded. Therefore, the analysis was conducted on the 69 subjects who completed the test.

Results

Blood uric acid levels measured in the 0th week and the 12th week are shown in Table 1.

TABLE 1

| | 0th Week | 12th Week |
|---|---|---|
| Blood uric acid level (mg/dl) | 6.5 ± 1.2 | 6.3 ± 1.2* |

The results are shown as mean value plus standard deviation.
*p < 0.05 vs 0th week value comparison

A Student's t-test showed a significant reduction in blood uric acid levels from the baseline at 12 weeks. This suggests that blood uric acid levels can be reduced or hyperuricemia can be prevented or ameliorated.

### Example 2

Preparation of Test Sample

The culture solution of Bifidobacterium breve MCC1274 (FERM BP-11175) was concentrated and dried to obtain viable bacterium dry powder. The viable bacterium dry powder was mixed with an excipient to obtain a test sample.

Test Subjects

Healthy subjects (with a BMI from 25 to 30) who were aged 20 to 65 when consent was obtained were registered as test subjects. In addition, 40 persons who did not violate exclusion criteria (1) to (7) below were selected based on body composition measurements, blood tests, and physician interviews. The average age of the subjects was 45.4±9.8 years.

(1) Persons with a medical history including treatment for serious disease

(2) Persons who suffer from and take medicine for a gastrointestinal disease

(3) Persons receiving medical treatment for lifestyle-related disease (diabetes, high blood pressure, dyslipidemia)

(4) Persons with drug allergies or serious food allergies

(5) Persons who are pregnant, intending to become pregnant during the trial period, or are breastfeeding

(6) Persons who are heavy smokers, heavy drinkers and/or have erratic life habits

(7) Persons deemed to be unsuitable as a test subject by the examining doctor or assisting doctor based on the subject's background, physical findings, or the interview.

Testing Methodology

After a two-week observational period, the subjects ingested the test sample once daily with water 30 minutes after a meal for 12 consecutive weeks. The daily intake of the test group was at least 20 billion viable Bifidobacterium breve MCC1274 (FERM BP-11175) bacteria.

The subjects were given blood tests before taking the test sample (baseline: 0 weeks) and at 12 weeks to measure blood uric acid concentrations (mg/dL). A review was conducted to exclude persons who did not meet analysis criteria established in advance (namely, those who had an ingestion rate of less than 80%, those who violated the medicine dose or ingested prohibited foods, and those who were found to have violated or deviated significantly from the test plan). However, none of them were excluded. Therefore, the analysis was conducted on the 40 subjects who completed the test.

Results

Blood uric acid levels measured in the 0th week and the 12th week are shown in Table 2.

TABLE 2

| | 0th Week | 12th Week |
|---|---|---|
| Blood uric acid level (mg/dl) | 6.9 ± 1.3 | 6.6 ± 1.1* |

The results are shown as mean value plus standard deviation.
*p < 0.05 vs 0th week value comparison

A Student's t-test showed a significant reduction in blood uric acid levels from the baseline at 12 weeks. This suggests that blood uric acid levels can be reduced or hyperuricemia can be prevented or ameliorated.

### PRODUCTION EXAMPLES

The following are production examples of pharmaceutical and food or beverage compositions for reducing blood uric acid levels or preventing or ameliorating hyperuricemia.

### Production Example 1

Bifidobacterium breve MCC1274 (FERM BP-11175) is added to 3 mL of MRS liquid medium, and the solution is anaerobically cultured at 37° C. for 16 hours, concentrated, and freeze-dried to obtain a freeze-dried powder of the bacteria (bacterial powder). The bacterial powder is then uniformly mixed with whey protein concentrate (WPC) and prebiotics (lacturose, raffinose, and galactooligosaccharides) to obtain a composition. Next, 20 g of the composition is dissolved in 200 g of water to obtain a composition for reducing blood uric acid levels or preventing or ameliorating hyperuricemia. Administration of this composition can reduce blood uric acid levels or prevent or ameliorate hyperuricemia.

### Production Example 2

Bifidobacterium breve MCC1274 (FERM BP-11175) is added to 3 mL of MRS liquid medium, and the solution is anaerobically cultured at 37° C. for 16 hours, concentrated, and freeze-dried to obtain a freeze-dried powder of the bacteria (bacterial powder). The bacterial powder is then uniformly mixed with a dry powder of milk protein concentrate (MPC 480 from Fontera, protein content 80 wt %, casein protein:whey protein=approx. 8:2) and prebiotics (lacturose, raffinose, and galactooligosaccharides) to obtain a composition. Next, 20 g of the composition is dissolved in 200 g of water to obtain a composition for reducing blood uric acid levels or preventing or ameliorating hyperuricemia. Administration of this composition can reduce blood uric acid levels or prevent or ameliorate hyperuricemia.

### Production Example 3

Bifidobacterium breve MCC1274 (FERM BP-11175) is added to 3 mL of MRS liquid medium, and the solution is anaerobically cultured at 37° C. for 16 hours, concentrated, and freeze-dried to obtain a freeze-dried powder of the bacteria (bacterial powder). Next, prebiotics (lacturose, raffinose and galactooligosaccharides) and crystalline cellulose are placed in a stirring granulator and mixed together. Afterwards, purified water is added to granulate, and the granulate is dried to obtain a granulate (pharmaceutical composition) containing bacterial extract, prebiotics, and an excipient. Administration of this composition can reduce blood uric acid levels or prevent or ameliorate hyperuricemia.

### Production Example 4

The following is a method used to produce a fermented milk product containing Bifidobacterium breve MCC1274 (FERM BP-11175).

First, raw milk, water as needed, and other components are mixed together, and homogenization and heat sterilization are preferably performed. Homogenization and heat sterilization can be performed in the usual manner. A lactic acid bacteria starter is added (inoculated) to the heat-sterilized milk preparation, and fermentation is carried out by maintaining the temperature at a predetermined fermentation temperature to obtain a fermented product. The fermentation forms curds.

The lactic acid bacteria starter can use any lactic acid bacteria commonly used in yogurt such as Lactobacillus bulgaricus, Lactococcus lactis, or Streptococcus thermophilus. When the pH reaches the target value, the curds are broken up by stirring and cooled to 10° C. or lower to obtain a fermented product. By cooling to 10° C. or lower, the activity of the lactic acid bacteria can be reduced to suppress the formation of acid.

Next, the fermented product obtained in the fermentation step is subjected to heat treatment to obtain a heated fermented product (heat-treated fermented product). By heating the fermented product sufficiently, production of acid by lactic acid bacteria can be prevented in the heated fermented product. In this way, any decrease in pH can be suppressed during the subsequent production steps and/or during storage of the concentrated fermented milk product containing Bifidobacterium. This improves the survival rate of the Bifidobacterium.

Next, Bifidobacterium breve MCC1274 (FERM BP-11175) and prebiotics (lacturose, raffinose and galactooligosaccharides) are added to the fermented product obtained in the heat treatment step. The amount of Bifidobacterium breve MCC1274 (FERM BP-11175) added to the heated fermentation product is preferably from 1×10⁷ to 1×10¹¹ cfu/ml and more preferably from 1×10⁸ to 1×10¹⁰ cfu/ml. In the case of dead Bifidobacterium breve MCC1274 (FERM BP-11175), cfu/ml can be converted to cells/ml.

After heating, Bifidobacterium breve MCC1274 (FERM BP-11175) and prebiotics are added to the fermented product, which is then concentrated. The concentration step can be performed using any concentration method common in the art. For example, centrifugation or membrane separation can be used. In the centrifugation method, whey is removed from the concentrate (heated fermented product containing added bifidobacteria and prebiotics) to increase the amount of concentrated fermented milk containing bifidobacteria and prebiotics in terms of solid content.

Administration of this fermented milk product can reduce blood uric acid levels or prevent or ameliorate hyperuricemia.

### Production Example 5

The following is a method used to produce an infant formula containing Bifidobacterium breve MCC1274 (FERM BP-11175).

Here, 10 kg of desalted milk whey protein powder (from Mirai), 6 kg of milk casein powder (from Fonterra), 48 kg of lactose (from Mirai), 920 g of a mineral mixture (from Tomita Pharmaceuticals), 32 g of a vitamin mixture (from Tanabe Seiyaku), 500 g of lactulose (from Morinaga Milk Industries), 500 g of raffinose (from Nippon Kanso Sugar), and 900 g of galacto-oligosaccharide liquid sugar (from Yakult Pharmaceutical Industries) are heat-dissolved in 300 kg of hot water at 90° C. for 10 minutes, 28 kg of a prepared fat (from Taiyo Yushi Corporation) is added, and the solution is homogenized. Afterwards, sterilization, concentration, and spray-drying are performed to prepare about 95 kg of infant formula powder. Next, 100 g of Bifidobacterium breve MCC1274 (FERM BP-11175) powder (1.8×10¹¹ cfu/g from Morinaga Milk) dispersed in starch is added to prepare about 95 kg of infant formula powder containing Bifidobacterium and oligosaccharides. When the resulting powder is dissolved in water to obtain liquid infant formula with a standard solid concentration of 14% (w/v), the Bifidobacterium count in the liquid infant formula is 2.7×10⁹ cfu/100 mL.

Administration of this infant formula can reduce blood uric acid levels or prevent or ameliorate hyperuricemia.

1. A composition for lowering blood uric acid levels comprising Bifidobacterium breve as an active ingredient.

2. A composition for preventing or ameliorating hyperuricemia comprising Bifidobacterium breve as an active ingredient.

3. A composition according to claim 1, wherein the Bifidobacterium breve is Bifidobacterium breve MCC1274 (FERM BP-11175).

4. A composition according to claim 1, wherein the composition is a pharmaceutical composition.

5. A composition according to claim 1, wherein the composition is a food or beverage composition.

6. (canceled)

7. (canceled)

8. (canceled)

9. (canceled)

10. A method of lowering blood uric acid level in a subject comprising administering Bifidobacterium breve to the subject.

11. A method according to claim 10, wherein the Bifidobacterium breve is Bifidobacterium breve MCC1274 (FERM BP-11175).

12. A method of preventing or ameliorating hyperuricemia in a subject comprising administering Bifidobacterium breve to the subject.

13. A method according to claim 12, wherein the Bifidobacterium breve is Bifidobacterium breve MCC1274 (FERM BP-11175).
